(19) United States
(12) Patent Application Publication
(10) Pub. No.: US 2024/0245574 A1
(43) Pub. Date: Jul. 25, 2024

(54) THERAPEUTIC SPECTACLES FOR RELIEVING DRY EYES

(71) Applicant: EYEDETEC MEDICAL, INC., Danville, CA (US)

(72) Inventors: Vance THOMPSON, SIOUX FALLS, SD (US); Barry J. LINDER, Scottsdale, AZ (US)

(73) Assignee: EYEDETEC MEDICAL, INC., Danville, CA (US)

(21) Appl. No.: 18/561,537

(22) PCT Filed: May 26, 2022
(86) PCT No.: PCT/US2022/031189

§ 371 Date: Nov. 16, 2023

Related U.S. Application Data
(60) Provisional application No. 63/195,333, filed on Jun. 1, 2021.

(51) Int. Cl. A61H 23/02; A61F 9/02

(52) U.S. Cl. CPC A61H 23/0218; A61F 9/029; A61H 2201/10; A61H 2201/165; A61H 2201/501; A61H 2201/5097; A61H 2205/023; A61H 2230/00

(57) ABSTRACT

A therapeutic spectacles device for reliving symptoms of dry eye disease. The device includes a protective housing configured to house a stimulus mechanism and an internal power source; at least one contact pad attached to the protective housing; and an attachment mechanism arranged on the protective housing. The at least one contact pad is configured, to contact a targeted area within a region selected from patient's nasofrontal region, supraorbital region, suborbital region, temporal region, or any combination of the same. The attachment mechanism can be configured to fix the protective housing to the therapeutic spectacles device such that the at least one contact pad rests against the targeted, area, when the therapeutic spectacles device is fixed on spectacles worn by the patient. The stimulus mechanism can be configured to deliver a stimulus through the at least one contact pad to the targeted area.

### CROSS REFERENCE TO RELATED APPLICATIONS

This application is related to, and claims the priority to, U.S. Provisional Patent Application Ser. No. 63/195,333, entitled, “THERAPEUTIC SPECTACLES FOR RELIEVING DRY EYES,” filed on Jun. 1, 2021, which is hereby incorporated by reference in its entirety into this application.

### TECHNICAL FIELD

Methods and devices for treating dry eyes are provided; more particularly, methods and devices for treating meibomian gland dysfunction in humans using one or more stimuli administered cutaneously to the nasofrontal region between the supra alar crease up to and including the glabella, and between the inner canthi.

### BACKGROUND

Dry eye disease is an eye condition that affects a person's quality of life by causing discomfort of the eyes and sometimes affecting a patient's vision. The disease can be caused by chronically obstructed or abnormal meibomian glands, which are sebaceous glands of the eyelids that secrete an oily substance called meibum that helps prevent evaporation of the eye's tear film. When meibomian glands become obstructed or otherwise secrete less meibum, the tear film evaporates more readily, resulting in a chronic condition called meibomian gland dysfunction. This condition can be worsened when the affected person spends a significant amount of time using video screens (e.g., computer screens, tablets, smart phones, video games, etc.), which can cause the patient to blink less frequently. By blinking less frequently, tear film evaporation increases, which can increase inflammation, which can affect the proper functioning of the meibomian glands, thus worsening the overall condition.

Conventional treatments for meibomian gland dysfunction include simple at-home remedies, such as periodically resting a warm wash cloth over the eyes for several minutes or gently massaging the affected area with the finger tips. However, these methods are not always effective and some patients still require additional treatment. More complex clinical treatments that require close professional monitoring are also available, such as corticosteroid therapy and antibiotics. These treatments also may not provide complete relief, and can be unsafe in some circumstances.

### SUMMARY

This disclosure provides new and effective methods and devices for relieving symptoms of dry eye disease, particularly resulting from meibomian gland dysfunction. These methods and devices can be adapted so that they can be safely used by the average person at home, in the office, or anywhere eye glasses can be worn.

Embodiments of therapeutic spectacles devices for rehabilitating a patient's tear film by inducing tear production and blinking are described. A therapeutic spectacles device can include: a protective housing that houses a stimulus mechanism and an internal power source; at least one contact pad on the protective housing; and an attachment mechanism on the protective housing for attaching the therapeutic spectacles device to a pair of spectacles.

The at least one contact pad can be configured to rest against a targeted area within a region selected from the group consisting of a patient's nasofrontal region, supraorbital region, suborbital region, temporal region, and any combination of the same. The attachment mechanism can be configured to fix the protective housing to spectacles such that the at least one contact pad rests against the targeted area when the therapeutic spectacles device is fixed on the spectacles and the spectacles are worn by the patient. The internal power source can have an energy capacity sufficient to provide uninterrupted power to operate the therapeutic spectacles device continuously for a period of time. The stimulus mechanism can be electrically connected to and powered by the internal power source, and can be configured to deliver a stimulus through the contact pad to the targeted area.

The stimulus mechanism and contact pad can be positioned such that, when the therapeutic spectacles device is fixed on spectacles worn by the patient, the stimulus is delivered through the nasal bridge of the patient to the external branch of the patient's trigeminal cranial nerve to stimulate neuromodulation of the external branch of the patient's trigeminal cranial nerve. The stimulus mechanism can generate a vibration stimulus for stimulating the nasofrontal region. The position of the contact pad and stimulus mechanism can be positioned such that the vibration stimulus, when administered to the patient, has a wave direction that is perpendicular to the nasal bridge where it meets the external branch of the trigeminal cranial nerve. The stimulus mechanism can generate an electromagnetic radiation stimulus for stimulating the targeted area.

According to at least one embodiment, the therapeutic spectacles device can also include a logic mechanism housed in the housing, the logic mechanism configured to automatically switch the stimulus mechanism on and off under given conditions. The therapeutic spectacles device can also include a timing mechanism housed in the housing, the timing mechanism configured to automatically switch the stimulus mechanism off after a given period of time. According to at least one embodiment, the therapeutic spectacles device can include a controller housed in the housing, the controller being configured to control the stimulus mechanism using received input. According to at least one embodiment, the at least one contact pad of the therapeutic spectacles device can include at least two contact pads positioned to stimulate at least two targeted areas within the region selected from the group consisting of the patient's nasofrontal region, supraorbitala region, suborbital region, temporal region, and any combination of the same. In such an embodiment, the controller can be configured to control the stimulus mechanism such that the at least two targeted areas can be targeted for stimulus independently.

According to at least one embodiment, the therapeutic spectacles device can include at least one sensor, the sensor being configured to observe a physical condition associated with the patient's tear film (e.g., the patient's blink rate, tear break-up time, head movement, or tear meniscus).

According to at least one embodiment, the therapeutic spectacles can include a radio transmitter and receiver housed in the housing, the radio transmitter and receiver can be configured to wirelessly transmit and receive data from the therapeutic spectacles device to an external computing device, the external computing device can have an interface (e.g., a software application) for controlling the therapeutic spectacles device. According to at least one embodiment, the internal power source can be a rechargeable battery.

Embodiments of therapeutic spectacles for administering a therapeutic stimulus to induce tear production in a patient are also described, the therapeutic spectacles can include: a pair of unobstructed lenses set in a frame, the frame having a protective housing that houses a stimulus mechanism and an internal power source; and at least one contact pad attached to the frame. The contact pad can be configured so that when the therapeutic spectacles are worn by the patient, the contact pad rests against at least one targeted area in a region selected from the group consisting of a patient's nasofrontal region, supraorbital region, suborbital region, temporal region, or any combination of the same when the therapeutic spectacles are worn by the patient. The internal power source can have an energy capacity sufficient to provide uninterrupted power to operate the therapeutic spectacles continuously for a period of time. The stimulus mechanism can be electrically connected to and powered by the internal power source, and being configured to deliver a stimulus through the contact pad to the at least one targeted area.

According to at least one embodiment, the stimulus mechanism and contact pad are positioned such that, when the therapeutic spectacles are worn by the patient, the stimulus is delivered through the nasal bridge of the patient to the external branch of the patient's trigeminal cranial nerve to stimulate neuromodulation of the external branch of the patient's trigeminal cranial nerve.

According to at least one embodiment, the stimulus mechanism generates a vibration for stimulating the nasofrontal region. According to at least one embodiment, the position of the contact pad and stimulus mechanism are positioned such that the vibration, when administered to the patient, has a wave direction that is perpendicular to the nasal bridge where it meets the external branch of the trigeminal cranial nerve.

According to at least one embodiment, the stimulus mechanism can generate electromagnetic radiation for stimulating the nasofrontal region.

The therapeutic spectacles can also include a logic mechanism housed in the protective housing, and the logic mechanism can be configured to automatically switch the stimulus mechanism on and off under given conditions. According to at least one embodiment, the therapeutic spectacles also include a timing mechanism housed in the protective housing, the timing mechanism configured to automatically switch the stimulus mechanism off after a given period of time. According to at least one embodiment, the therapeutic spectacles can include a controller housed in the protective housing, and the controller can be configured to control the stimulus mechanism using a received input. According to at least one embodiment, the at least one contact pad can include at least two contact pads positioned to stimulate at least two targeted areas within the region selected from the group consisting of the patient's nasofrontal region, supraorbital region, suborbital region, temporal region, and any combination of the same. In such an embodiment, the controller can be configured to control the stimulus mechanism such that the at least two targeted areas can be targeted for stimulus independently.

According to at least one embodiment, the therapeutic spectacles can include at least one sensor. The sensor can be configured to observe a number of physical conditions associated with the patient's tear film (e.g., tear meniscus, blink rate, tear break-up time, head movement, etc.).

According to at least one embodiment, the therapeutic spectacles can also have a radio transmitter and receiver housed in the protective housing, and the radio transmitter and receiver can be configured to wirelessly transmit and receive data from the therapeutic spectacles to an external computing device, and vice versa. The external computing device can have an interface (e.g., software application) for controlling the therapeutic spectacles. The internal power source can be a rechargeable battery in some embodiments.

### BRIEF DESCRIPTION OF THE DRAWINGS

The embodiments of this disclosure will be understood by the following detailed description along with the accompanying drawings. The embodiments shown in the figures only illustrate several embodiments of the disclosure. The disclosure admits of other embodiments not shown in the figures, and is not limited to the contents of the illustrations.

FIG. 1 is an anatomical illustration of the external branch of a person's trigeminal cranial nerve.

FIG. 2 is an illustration of an embodiment of a therapeutic spectacles device.

FIG. 3 is an illustration of an embodiment of therapeutic spectacles.

FIG. 4 is an illustration of an embodiment of therapeutic spectacles configured to stimulate more than one target area.

FIG. 5 is an illustration of an embodiment of a therapeutic mask device.

### DETAILED DESCRIPTION

This disclosure describes various embodiments related to methods and devices for rehabilitating a patient's tear film by providing a therapeutic stimulus to induce blinking and tear formation. Embodiments include spectacles having a built-in mechanism(s) for administering a stimulus to a nasofrontal region of the patient's face, therapeutic devices designed to be attached and worn with spectacles, therapeutic masks, and methods of using the same.

In this disclosure the term “spectacles” means any of various devices resembling a pair of glasses, which can be worn on one's face and looked through, such as eye glasses, sunglasses, pince-nez, computer eye glasses, goggles, virtual reality headset, augmented reality headset, mixed reality headset, etc.

In this disclosure the term “nasofrontal region” refers to the region of a patient's face that includes nasal and frontal anatomical features. Particularly, the region of a patient's face between the supra alar crease up to and including the glabella, and between the patient's inner canthi. Nonlimiting examples of anatomical features included in the nasofrontal region include the nasal bridge, glabella, portions of the nose, etc.

In this disclosure the term “supraorbital region” refers to the region of a patient's face above the patient's eye. For example, the supraorbital region can be the region between a patient's upper eyelid crease up to and including the forehead, and more particularly up to and including the supraorbital ridge.

In this disclosure the term “suborbital region” refers to the region of a patient's face below the patient's eye. For example, the suborbital region can be the zygomaticomaxillary region of a patient's face below the patient's lower eyelid crease.

In this disclosure the term “temporal region” refers to the lateral region of a patient's head behind the orbits. For example, the region between the outer canthus to the mastoid part of the temporal bone. Nonlimiting examples of anatomical features included in the temporal region include the sphenoidal region, temporal region, and certain zygomatic features.

The anatomical terms used in this disclosure refer to human anatomy, but the terms are used in a manner that one of ordinary skill could identify comparable anatomical features and regions in many animals. While the methods and devices described in this disclosure are particularly suitable for human patients, it will also be apparent to one of ordinary skill that such methods and devices can be adapted for animals having eyes, eyelids, and a blinking reflex substantially similar to that of humans.

The act of viewing a video screen can cause one to blink less frequently and exacerbate certain conditions that cause dry eyes, the devices and methods described in this disclosure can be particularly useful for persons who spend significant periods of time viewing video screens. At least one advantage of the devices and methods is that they can be adapted so that they are safe for at-home use, making them more convenient than some clinical treatments. In some embodiments, the therapeutic devices can have simple controls or can be controlled using a software application having a familiar interface, such as an application for a smart phone, making the device accessible for many users who lack professional training. Because these devices are more accessible than other clinical treatments, they are more likely to be used more frequently, increasing their effectiveness. In some embodiments, the patient's view can be substantially unobstructed by the therapeutic device so that the patient can simultaneously carry out other activities that require an unobstructed view.

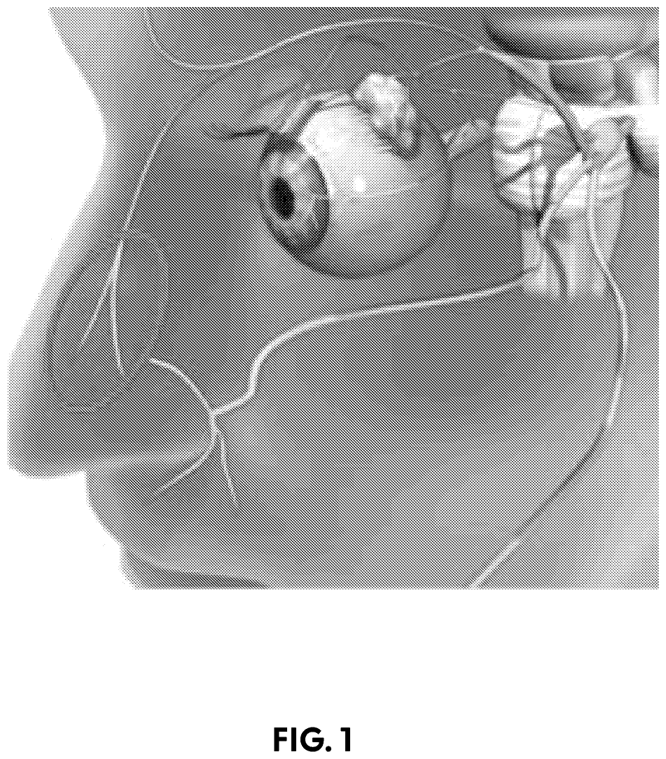

Stimulating neuromodulation of the external branch of the trigeminal cranial nerve, such as by mechanical or electromagnetic stimulation, can activate the lacrimal function unit to increase tear production, goblet cell mucin excretion, and meibomian gland expression. Moreover, cutaneous stimulation, such as by mechanical or electromagnetic stimulus, of the nasofrontal region between the supra alar crease of the patient's nose up to and including the patient's glabella, and between the patient's inner canthi can stimulate one's blink reflex. This is particularly the case for stimulation of the nasal bridge and glabella. Stimulation causing neuromodulation of the ophthalmic nerve in the region between the upper eyelid crease up to and including the forehead, or more particularly up to and including the supraorbital ridge, can also induce blinking. Similarly, stimulation of the suborbital region can cause increased blinking. Stimulation of the sphenoidal and temporal regions can also cause one to blink. An increase in blink rate and tear production can help provide relief for symptoms of dry eye disease and can help rehabilitate the tear film of patients experiencing meibomian gland dysfunction in some cases. FIG. 1 is an anatomical illustration showing the general position of the external branch of the trigeminal cranial nerve.

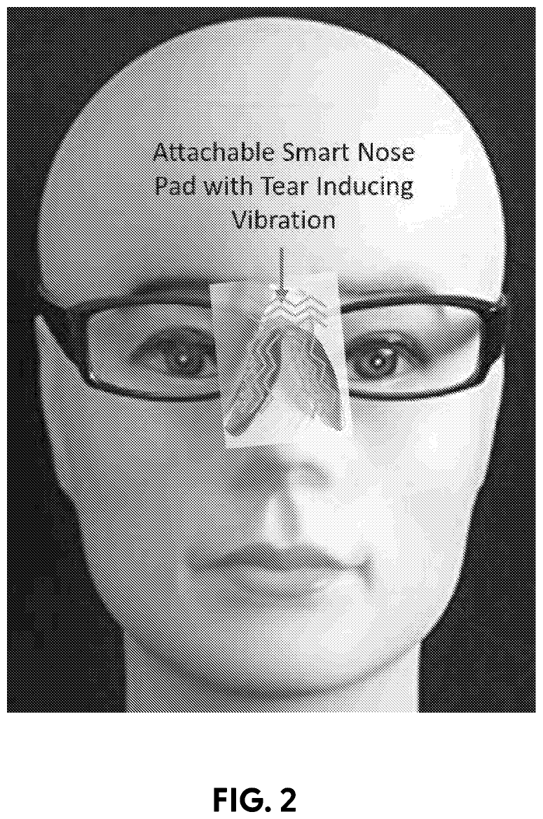

Devices for administering cutaneous stimulation to stimulate neuromodulation of the external branch of the trigeminal cranial nerve and/or stimulating blink reflex can include an attachment to be used with spectacles, referred to here as a therapeutic spectacles device. FIG. 2 shows an illustration of an embodiment of a therapeutic spectacles device. The therapeutic spectacles device can include a protective housing that houses a stimulus mechanism and an internal power source. Preferably, the protective housing encloses the components it houses. The protective housing can be made of a material, such as plastic, metal, carbon fiber, etc. In some embodiments, the protective housing can be dimensioned to fit the contours of a targeted area within the patient's nasofrontal region. For example, the protective housing can be dimensioned to fit over the patient's nasal bridge such that the protective housing fits the contours of the patient's nasal bridge and can rest upon it. According to at least one embodiment, the protective housing can be dimensioned so that it fits the contours of a targeted area within the supraorbital region of the patient's face. For example, the protective housing can be dimensioned to fit the contours of a targeted area within the region between the upper eyelid up to and including the forehead, or more particularly up to and including the supraorbital ridge. According to at least one embodiment, the protective housing can be dimensioned to fit the contours of a targeted area within the suborbital region of the patient's face. For example, the protective housing can be dimensioned to fit the contours of a targeted area within the zygomaticomaxillary region below the patient's lower eye lid. According to at least one embodiment, the protective housing can be dimensioned to fit the contours of a targeted area within the temporal region of the patient's head.

In some embodiments, the protective housing can be dimensioned so that, when attached to spectacles worn by the patient, it does not significantly obscure the patient's vision. By way of example and not limitation, if the therapeutic spectacles device is fixed to spectacles and positioned to stimulate a targeted area in the nasofrontal region the protective housing can be dimensioned so that it does not extend beyond the patient's nose and does not extend beyond the patient's inner canthi. In another example, if the therapeutic spectacles device is fixed to spectacles and positioned to stimulate a targeted area within the supraorbital region of the patient's face, the protective housing can be dimensioned so that it does not extend below the patient's upper eyelid crease. Similarly, if the therapeutic spectacles device is fixed to spectacles and positioned to stimulate a targeted area within the suborbital region of the patient's face, the protective housing can be dimensioned so that it does not extend above the patient's lower eyelid crease. In this manner, the therapeutic spectacles device can be used by the patient while the patient simultaneously performs other activities that require unobscured vision (e.g., reading, walking, bird watching, etc.) making the treatment more enjoyable for the patient. The experience for the patient is nearly transparent in the sense that the patient can carry out everyday tasks while simultaneously receiving therapeutic treatment from the therapeutic spectacles device without its interference and without the patient's continuing active effort. The patient is more likely to continue using the therapeutic spectacles device because of this transparency, in this way increasing its effectiveness.

The therapeutic spectacles device can include at least one contact pad, preferably at least two contact pads, in some embodiments at least three contact pads, in some embodiments at least four contact pads, in some embodiments at least five contact pads. In some embodiments, the therapeutic spectacles device can have between one and nine contact pads. According to at least one embodiment, the therapeutic spectacles device can have at least one contact pad positioned to contact a targeted area of the patient's face selected from the group comprising the patient's nasofrontal region, supraorbital region, suborbital region, temporal region, or any combination of the same.

According to at least one embodiment, the therapeutic spectacles device can include at least one contact pad for contacting a targeted area within the patient's nasofrontal region. Preferably at least two contact pads for contacting either side of the nasal bridge. According to at least one embodiment, the therapeutic spectacles device can include at least three contact pads, two for contacting either side of the nasal bridge and one for contacting the region between the two sides of the nasal bridge (e.g., the nasion, glabella, etc.). According to at least one embodiment, the therapeutic spectacles device can have at least one contact pad for contacting a targeted area within the supraorbital region of the patient's face. For example, the therapeutic spectacles device can have at least one contact pad positioned to contact the patient's supraorbital ridge.

According to at least one embodiment, the therapeutic spectacles device can have at least one contact pad for contacting a targeted area of the patient's face within the suborbital region of the patient's face. For example, the therapeutic spectacles device can have at least one contact pad positioned to contact a targeted area of the zygomaticomaxillary region below the patient's lower eyelid crease.

According to at least one embodiment, the therapeutic spectacles device can have at least one contact pad for contacting a targeted area within the temporal region of the patient's head. For example, the therapeutic spectacles device can have at least one contact pad for contacting a targeted area between the patient's outer canthus and the patient's mastoid.

The at least one contact pad can be attached to the protective housing, such as by being integrated with the protective housing or attached to the protective housing by a number of means (e.g., by pad arms that extend from the protective housing to the at least one contact pad, adhesion, fastening, etc.). According to at least one embodiment, the at least one contact pad can be a surface of the protective housing. According to at least one embodiment, the position of the at least one contact pad can be adjustable by the user. According to at least one embodiment, the at least one contact pad can apply pressure over the nasal bridge by mechanical force to enhance stimulus of the targeted nerves.

The therapeutic spectacles device can include an attachment mechanism on the protective housing for attaching the therapeutic spectacles device to a pair of spectacles. Examples of suitable attachment mechanisms can include clasps, fasteners, magnetic mounts, interlocking mounts, etc. The attachment mechanism can be designed so that the therapeutic spectacles can be attached and removed from the spectacles without damaging them. In some embodiments, the attachment mechanism can be designed and configured to attach to the frame, bridge, contact pad, pad arm of the spectacles, or any combination of the same. The attachment mechanism can be configured to fix the protective housing to the spectacles such that the at least one contact pad rests against the nasofrontal region when the therapeutic spectacles device is fixed on spectacles worn by the patient.

The internal power source can have an energy capacity sufficient to provide uninterrupted power to operate the therapeutic spectacles device continuously for a period of time. For example, the internal power source can be a rechargeable battery, such as a nickel-cadmium battery or a lithium-ion battery. In some embodiments, the internal power source can be a rechargeable battery. In some embodiments, the internal power source can be a capacitor. It is also contemplated that the internal power source can include a generator such as a photovoltaic cell or thermoelectric generator for converting the patient's body heat to electricity.

The stimulus mechanism can be electrically connected to and powered by the internal power source and can be configured to deliver a stimulus through the contact pad to a targeted area within the patient's nasofrontal region, supraorbital region, suborbital region, temporal region, or any combination of the same. By way of example and not limitation, the stimulus mechanism and contact pad can be positioned such that, when the therapeutic spectacles device is fixed on spectacles worn by the patient, the stimulus is delivered through the nasal bridge of the patient to the external branch of the patient's trigeminal cranial nerve to stimulate neuromodulation of the external branch of the patient's trigeminal cranial nerve. In some embodiments, the stimulus mechanism can generate a mechanical stimulus, such as by vibration, or oscillatory motion, for stimulating the targeted area. When a vibration stimulus is used to target a targeted area within the nasofrontal region, preferably the contact pad and stimulus mechanism can be positioned such that the vibration stimulus has a wave direction that is perpendicular to the nasal bridge where it meets the external branch of the trigeminal cranial nerve. According to at least one embodiment, the frequency and amplitude of the vibration can be optimized to stimulate the external branch of the trigeminal cranial nerve. In some embodiments, the stimulus mechanism can generate an electromagnetic radiation stimulus, such as intense pulsed light, for stimulating the targeted area. In some embodiments the electromagnetic radiation stimulus can be polychromatic light having a wavelength from 500-1,200 nanometers. According to at least one embodiment, the stimulus mechanism can generate an electrical impulse stimulus for stimulating the targeted area.

According to at least one embodiment, the therapeutic spectacles device can also include a logic mechanism housed in the housing, the logic mechanism configured to automatically switch the stimulus mechanism on and off under given conditions. In some embodiments, the logic mechanism can be a simple on-off switch. According to at least one embodiment, the logic mechanism can be a digital or analog logic gate. The therapeutic spectacles device can also include a timing mechanism housed in the housing, the timing mechanism can be a digital or analog mechanism configured to automatically switch the stimulus mechanism off after a given period of time. According to at least one embodiment, the therapeutic spectacles device can include a controller housed in the housing, the controller can be configured to control the stimulus mechanism using received input, such as by analog controls or by digital commands. According to at least one embodiment, the therapeutic spectacles device can include at least one sensor, the sensor can be configured to observe a physical condition associated with the patient's tear film (e.g., the patient's blink rate, tear break-up time, head movement, or tear meniscus). By way of example and not limitation, the at least one sensor can be a camera for observing the patient's tear meniscus, an accelerometer for measuring head movement, a sensor for detecting eyelid movement from blinking, such as radar or video, etc.

According to at least one embodiment, the therapeutic spectacles device can be configured to stimulate at least two targeted areas within the regions selected from the patient's nasofrontal region, supraorbital region, suborbital region, temporal region, or any combination of the same and can have a controller configured to control the stimulus mechanism such that each of the at least two targeted areas can be targeted for stimulus independently.

According to at least one embodiment, the therapeutic spectacles can include a radio transmitter and receiver housed in the housing, the radio transmitter and receiver can be configured to wirelessly transmit and receive data from the therapeutic spectacles device to an external computing device, the external computing device can have an interface (e.g., a software application) for controlling the therapeutic spectacles device. The interface can be used to allow the user to input information to the computing device so that calculations can be performed to optimize device performance and therapeutic results, and to display useful information to the user. By using a familiar interface, such as a graphical user interface for a smart phone application, complex processes and equations can be made more accessible to general users who may lack professional training. Data received by the computing device can be sent to a cloud application, where it can be stored and analyzed. An application protocol interface can be used to deliver data from the therapeutic spectacles device for use with other applications.

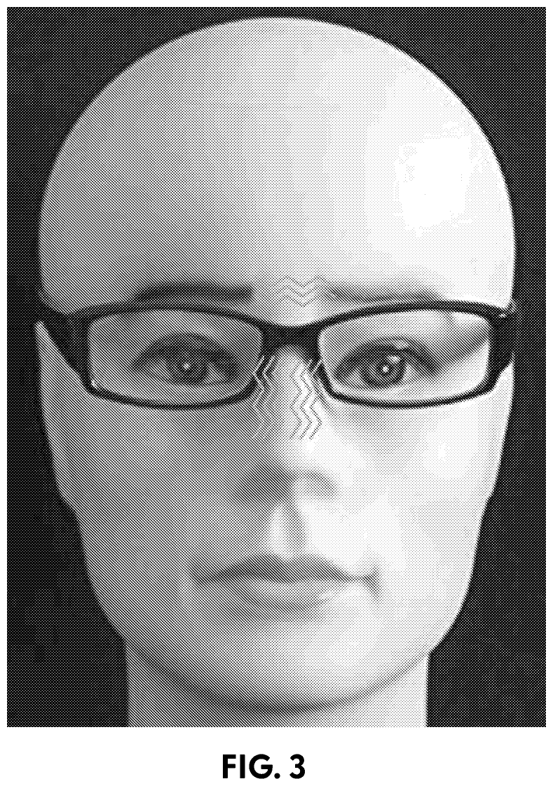

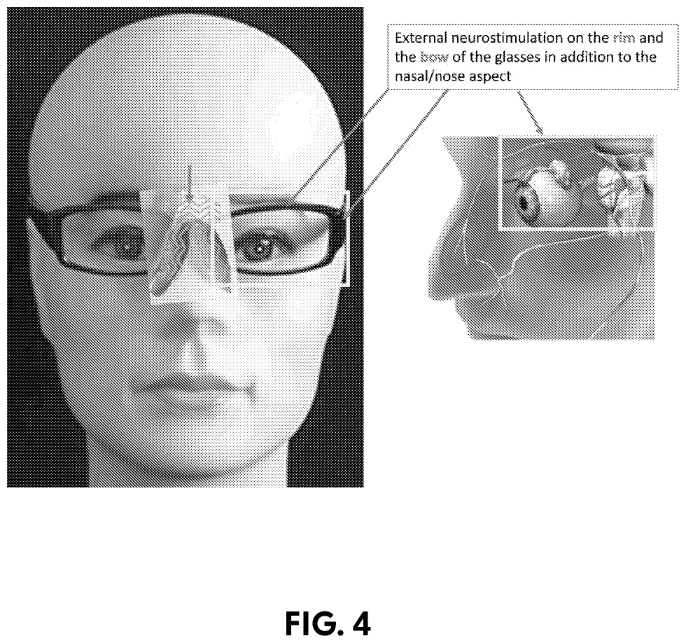

Embodiments of therapeutic spectacles for administering a therapeutic stimulus to induce tear production in a patient are also described. FIG. 3 shows an illustration of an embodiment of therapeutic spectacles that are designed to relieve symptoms of dry eye disease. The term “therapeutic spectacles” as used in this disclosure means a device having a pair of unobstructed lenses set in a frame, the frame having an integrated protective housing that houses a stimulus mechanism and an internal power source; and at least one contact pad attached to the frame. Here, the term “frame” refers to the arrangement of parts exclusive of the lenses that give the spectacles their structure. FIG. 4 shows an illustration of therapeutic spectacles having stimulus mechanisms housed in the frame (i.e., the bridge, rim, and bow) of the spectacles. Similar to the at least one contact pad of the therapeutic spectacles device described previously, the contact pad can be configured so that when the therapeutic spectacles are worn by the patient, the contact pad rests against a targeted area within the patient's nasofrontal region, supraorbital region, suborbital region, temporal region, or any combination of the same. The internal power source and stimulus mechanism can be configured similar to like components used in the therapeutic spectacles device described previously. According to at least one embodiment, the therapeutic spectacles can include a logic mechanism, timing mechanism, radio receiver and transmitter, user interface, cloud storage and analyzing of data, and application protocol interface similar to like components or features described previously.

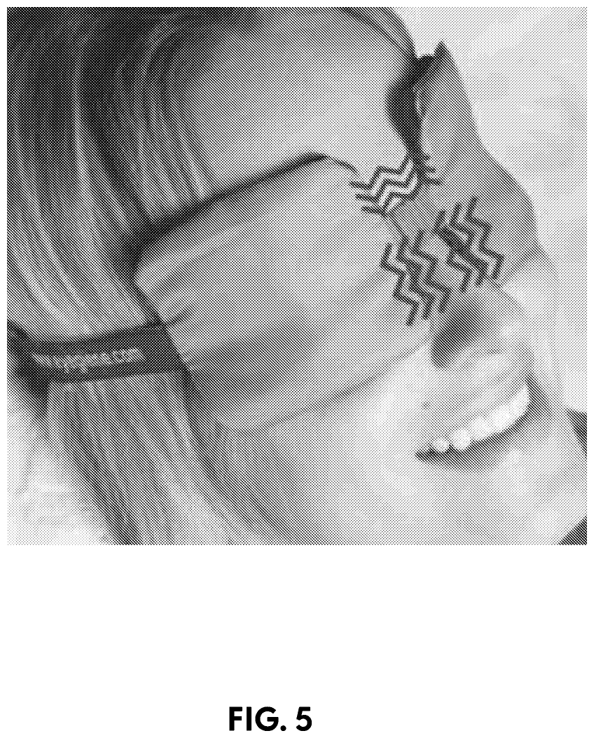

Embodiments of therapeutic masks for administering a therapeutic stimulus to induce tear production in a patient are also described. FIG. 5 shows an illustration of an embodiment of a therapeutic mask. As used in this disclosure, the term “therapeutic mask” refers to a mask that substantially obstructs one's vision when worm, and that has an integrated stimulus mechanism and an internal power source. The stimulus mechanism and internal power source can be configured similar to like components described previously. According to at least one embodiment, the therapeutic mask can have a compartment (e.g., pocket, envelope, opening, recess, chamber, receptacle, container, etc.) that houses the stimulus mechanism. According to at least one embodiment, the stimulus mechanism can be positioned to provide targeted stimulus of a targeted area within the patient's nasofrontal region, supraorbital region, suborbital region, temporal region, or any combination of the same. According to at least one embodiment, the therapeutic mask can be made from a material suitable to retain moisture. According to at least one embodiment, the compartment of the therapeutic mask can be substantially impervious to water such that the mask can be moistened without affecting its electronic functions during normal use. The therapeutic mask can be worn by the patient to relieve nighttime dry eye.

This disclosure can use the phrases “in some embodiments,” “in various embodiments,” “in an embodiment,” “at least one embodiment,” or “in embodiments,” which can each refer to one or more of the same or different embodiments. Furthermore, the terms “comprising,” “including,” “having.” and the like, as used with respect to embodiments of the present disclosure, are synonymous.

For certain embodiments, many details are provided for thorough understanding of the various components or steps. In other instances, well-known processes, devices, compositions, and systems are not described in particular detail so that the embodiments are not obscured by detail. Likewise, illustrations of the various embodiments can omit certain features or details so that various embodiments are not obscured.

In this disclosure and the appended claims, unless otherwise indicated, all numbers expressing quantities, percentages or proportions, and other numerical values used in the specification and claims are to be understood as being modified in all instances by the term “about.” The term “about” applies to all numeric values, whether or not explicitly indicated. This term generally refers to a range of numbers that one of ordinary skill in the art would consider a reasonable amount of deviation to the recited numeric values.

The drawings provide an illustration of certain embodiments. Other embodiments can be used, and logical changes can be made without departing from the scope of this disclosure. This disclosure is intended to disclose certain embodiments with the understanding that many other undisclosed changes and modifications can fall within the spirit and scope of the disclosure. The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal language of the claims.

Ordinal numbers (such as “first,” “second,” “third,” and so on), when used in this disclosure as an adjective before a term, merely identify a particular component, feature, step, etc. unless expressly otherwise provided. At times, ordinal numbers may be used to distinguish a particular feature from another feature that is described by the same term or similar term. Unless expressly otherwise provided, ordinal numbers do not indicate any relationship, order, quality, ranking, importance, or characteristic between features, components, steps, etc. Moreover, ordinal numbers do not define a numerical limit to the features, components, or steps identified by the ordinal number(s).

Ranges can be expressed in this disclosure as from about one particular value and to about another particular value. With these ranges, another embodiment is from the one particular value and/or to the other particular value, along with all combinations within the range. When the range of values is described or referenced in this disclosure, the interval encompasses each intervening value between the upper limit and the lower limit as well as the upper limit and the lower limit and includes smaller ranges of the interval subject to any specific exclusion provided.

Unless otherwise defined, all technical and scientific terms used in this specification and the appended claims have the same meanings as commonly understood by one of ordinary skill in the relevant art.

Where a method comprising two or more defined steps is recited or referenced in this disclosure or the appended claims, the defined steps can be carried out in any order or simultaneously except where the context excludes that possibility.

1. A therapeutic spectacles device for relieving symptoms of dry eye disease, the therapeutic spectacles device comprising:
a protective housing configured to house a stimulus mechanism and an internal power source;
at least one contact pad attached to the protective housing, the at least one contact pad configured to rest against a targeted area within a region selected from the group consisting of a patient's nasofrontal region, supraorbital region, suborbital region, temporal region, and any combination of the same; and
an attachment mechanism arranged on the protective housing, the attachment mechanism configured to fix the protective housing to the therapeutic spectacles device such that the at least one contact pad rests against the targeted area when the therapeutic spectacles device is fixed on spectacles worn by the patient,
wherein the internal power source has an energy capacity sufficient to provide uninterrupted power to operate the therapeutic spectacles device continuously for a period of time; and
wherein the stimulus mechanism is electrically connected to and powered by the internal power source, and is configured to deliver a stimulus through the at least one contact pad to the targeted area.

2. The therapeutic spectacles device of claim 1, in which the stimulus mechanism and the at least one contact pad are positioned such that, when the therapeutic spectacles device is fixed on spectacles worn by the patient, the stimulus is delivered through a nasal bridge of the patient to an external branch of a patient's trigeminal cranial nerve to stimulate neuromodulation of the external branch of the patient's trigeminal cranial nerve.

3. The therapeutic spectacles device of claim 1, in which the stimulus mechanism is configured to generate a vibration for stimulating the targeted area.

4. The therapeutic spectacles device of claim 1, in which the at least one contact pad and stimulus mechanism are positioned such that the vibration, when administered to the patient, has a wave direction that is perpendicular to a nasal bridge of the patient where it meets an external branch of a patient's trigeminal cranial nerve.

5. The therapeutic spectacles device of claim 1, in which the stimulus mechanism is configured to generate electromagnetic radiation for stimulating the targeted area.

6. The therapeutic spectacles device of claim 1, further comprising:
a logic mechanism housed in the protective housing, the logic mechanism configured to automatically switch the stimulus mechanism on and off under given conditions.

7. The therapeutic spectacles device of claim 1, further comprising:
a timing mechanism housed in the protective housing, the timing mechanism configured to automatically switch the stimulus mechanism off after a given period of time.

8. The therapeutic spectacles device of claim 1, further comprising:
a controller housed in the protective housing, the controller being configured to control the stimulus mechanism using received input.

9. The therapeutic spectacles device of claim 1, in which the at least one contact pad comprises at least two contact pads and in which the at least two contact pads are positioned to stimulate at least two targeted areas within the region selected from the group consisting of the patient's nasofrontal region, supraorbital region, suborbital region, temporal region, and any combination of the same; and
the controller is configured to control the stimulus mechanism such that the at least two targeted areas can be targeted for stimulus independently.

10. The therapeutic spectacles device of claim 1, further comprising:
at least one sensor, the sensor configured to observe a physical condition associated with a patient's tear meniscus.

11. The therapeutic spectacles device of claim 1, further comprising:
at least one sensor, the sensor configured to observe a physical condition associated with a patient's blink rate.

12. The therapeutic spectacles device of claim 1, further comprising:
at least one sensor, the sensor configured to observe a physical condition associated with a patient's tear break-up time.

13. The therapeutic spectacles device of claim 1, further comprising:
at least one sensor, the sensor being configured to observe a physical condition associated with the patient's head movement.

14. The therapeutic spectacles device of claim 1, further comprising:
a radio transmitter and receiver housed in the protective housing, the radio transmitter and receiver configured to wirelessly transmit and receive data from the therapeutic spectacles device to an external computing device, the external computing device having an interface for controlling the therapeutic spectacles device.

15. The therapeutic spectacles device of claim 1, in which the internal power source comprises a rechargeable battery.

16. Therapeutic spectacles for relieving symptoms of dry eye disease, the therapeutic spectacles comprising:
a pair of unobstructed lenses set in a frame, the frame comprising a protective housing configured to house a stimulus mechanism and an internal power source; and
at least one contact pad attached to the frame, the at least one contact pad configured to rest against at least one targeted area in a region selected from the group consisting of a patient's nasofrontal region, supraorbital region, suborbital region, temporal region, or any combination of the same when the therapeutic spectacles are worn by the patient,
wherein the internal power source has an energy capacity sufficient to provide uninterrupted power to operate the therapeutic spectacles continuously for a period of time, and
wherein the stimulus mechanism is electrically connected to and powered by the internal power source, and is configured to deliver a stimulus through the at least one contact pad to the at least one targeted area.

17. The therapeutic spectacles of claim 16, in which the stimulus mechanism and contact pad are positioned such that, when the therapeutic spectacles are worn by the patient, the stimulus is delivered through a nasal bridge of the patient to an external branch of a patient's trigeminal cranial nerve to stimulate neuromodulation of the external branch of the patient's trigeminal cranial nerve.

18. The therapeutic spectacles of claim 16, in which the stimulus mechanism is configured to generate a vibration for stimulating the nasofrontal region.

19. The therapeutic spectacles of claim 16, in which the position of the at least one contact pad and stimulus mechanism are positioned such that the vibration, when administered to the patient, has a wave direction that is perpendicular to a nasal bridge of the patient where it meets an external branch of a patient's trigeminal cranial nerve.

20. The therapeutic spectacles of claim 16, in which the stimulus mechanism is configured to generate electromagnetic radiation for stimulating the at least one targeted area.

21. The therapeutic spectacles of claim 16, further comprising:
a logic mechanism housed in the protective housing, the logic mechanism configured to automatically switch the stimulus mechanism on and off under given conditions.

22. The therapeutic spectacles of claim 16, further comprising:
a timing mechanism housed in the protective housing, the timing mechanism configured to automatically switch the stimulus mechanism off after a given period of time.

23. The therapeutic spectacles of claim 16, further comprising:
a controller housed in the protective housing, the controller being configured to control the stimulus mechanism using received input.

24. The therapeutic spectacles of claim 16, in which the at least one contact pad comprises at least two contact pads and in which the at least two contact pads are positioned to stimulate at least two targeted areas within the region selected from the group consisting of the patient's nasofrontal region, supraorbital region, suborbital region, temporal region, and any combination of the same; and
the controller is configured to control the stimulus mechanism such that the at least two targeted areas can be targeted for stimulus independently.

25. The therapeutic spectacles of claim 16, further comprising:
at least one sensor, the sensor configured to observe a physical condition associated with a patient's tear meniscus.

26. The therapeutic spectacles of claim 16, further comprising:
at least one sensor, the sensor configured to observe a physical condition associated with a patient's blink rate.

27. The therapeutic spectacles of claim 16, further comprising:
at least one sensor, the sensor configured to observe a physical condition associated with a patient's tear break-up time.

28. The therapeutic spectacles of claim 16, further comprising:
at least one sensor, the sensor configured to observe a physical condition associated with a patient's head movement.

29. The therapeutic spectacles of claim 16, further comprising:
a radio transmitter and receiver housed in the protective housing, the radio transmitter and receiver configured to wirelessly transmit and receive data from the therapeutic spectacles device to an external computing device, the external computing device having an interface for controlling the therapeutic spectacles.

30. The therapeutic spectacles of claim 16, in which the internal power source comprises a rechargeable battery.
